(19) United States
(12) Patent Application Publication
(10) Pub. No.: US 2007/0225505 A1
(43) Pub. Date: Sep. 27, 2007

(54) Method for the Preparation of (2S, 3AR, 7AS)-Octahydro-III-Indole-2-Carboxylic Acid as Key Intermediate in the Preparation of Trandolapril by Reacting a Cyclohexyl Aziridine with a Dialkyl Malonate

(76) Inventor: Pau Cid, Barcelona (ES)

Correspondence Address: NIXON & VANDERHYE, PC, 901 NORTH GLEBE ROAD, 11TH FLOOR, ARLINGTON, VA, 22203, US

(21) Appl. No.: 10/580,610

(22) PCT Filed: Nov. 25, 2004
(86) PCT No.: PCT/EP04/13377

§ 371 Date: Feb. 12, 2007

(30) Foreign Application Priority Data
Nov. 25, 2003 (EP) 03257417.0

(51) Int. Cl. C07D 209/04

(52) U.S. Cl. 548/469

(57) ABSTRACT

A method for the synthesis of a compound of formula I as a mixture of enantiomers, 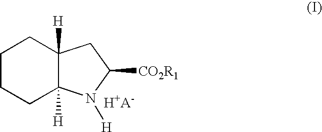
(wherein R₁ is H or an acid protective group and H⁺A⁻ indicates an optional acid with which the compound of formula I may form an ammonium salt) said method comprising; A) reacting a cyclohexyl aziridine with a dialkyl malonate, whereby to provide a trans-fused 3-alkylcarbonyl-octahydro-indol-2-one; B) decarbonylation at the 3-position, conversion of the ketone of the resulting trans-octahydro-indol-2-one to an optionally protected carboxylic acid group; and C) optionally removing any N-substitution if necessary.

The present invention relates to the synthesis of the cardiovascular drug trandolapril and in particular to improved synthetic methods for providing key stereochemical centres in trandolapril.

The angiotensin-converting enzyme (ACE) inhibitor trandolapril is commonly prescribed as a cardiovascular drug for the control and management of mild to severe hypertension (high blood pressure) and may be used alone or in combination with diuretics or other antihypertensive agents. Administration of trandolapril is typically oral at a level of around 0.5-4 mg once a day and may also be used in the management of conditions such as heart failure and left ventricular dysfunction following myocardial infarction.

Trandolapril itself is a prodrug, being converted to the acid form “trandolaprilat” in vivo. It is, however, generally desirable to prepare and administer the ester form. The structures of trandolapril and trandolaprilat are shown below.

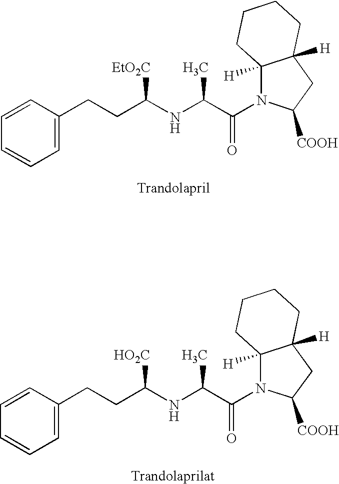

Various methods for the synthesis of trandolapril and related compounds have been proposed but each of these suffers from drawbacks. Frequently the syntheses require the use of dangerous reagents, which make industrial scale preparation hazardous and difficult and/or involve multiple steps resulting in a long and complex synthesis. One of the most important steps in the synthesis is the formation of the trans-fused octahydroindole ring, which is often difficult to separate from the cis-fused equivalent.

A number of the known synthetic routes to trandolapril proceed via the key intermediate (2S, 3aR, 7aS)-octahydro-1H-indole-2-carboxylic acid. This contains the key trans-fused octahydroindole ring and the correct stereochemistry for the carboxylic acid group at the 2-position. Frequently, these methods require the separation of the cis- and trans-fused rings and, in many cases, resolution of the carboxylate group at the 2-position is necessary. Where production of the trans-fused ring junction has been possible without generating significant quantities of the cis-product, the syntheses have been long and/or required dangerous reagents such as mercury compounds.

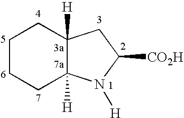

### (2S, 3aR, 7aS)-octahydro-1H-indole-2-carboxylic acid

U.S. Pat. No. 4,691,022 gives a synthesis of the above intermediate compound in relatively few steps but requires the trans-octahydroindole as the starting material. The result is also a mixture of the 2-α and 2-β compounds.

EP-A-084164/U.S. Pat. No. 4,933,361 provides an apparently effective method for the synthesis of the cis-fused intermediate beginning with the high-pressure hydrogenation of indole at 100 atmospheres of hydrogen and a platinum catalyst. This document also provides two methods for forming the trans-fused octahydroindole ring, but neither is indicated as being efficient. The first method provides the stereochemistry for the 2-position from substituted alanine, reacting this with activated cyclohexanone and cyclising the product to give a hexahydroindole. Unfortunately, the reduction of this hexahydroindole to the octahydro-compound produces both cis- and trans-fused product in unknown yield. The second method is to introduce the trans-ring via trans-octahydro-1H-quinolin-2-one, but no indication of yield in the key step is given and complex series of halogenation, partial re-hydrogenation and re-arrangement are required to reach the desired intermediate.

WO 00/40555/U.S. Pat. No. 6,559,318 relies on enzymic resolution of a 2-(2′,2′-methoxyethyl)cyclohexamine with Novozyme7 over 25 hours to provide the N-acetylated (1R, 2S) enantiomer which must then be separated by column chromatography from the unreacted (1S, 2R) enantiomer. Neither the enzymic resolution nor the chromatography steps are well suited to industrial scale preparations. There are also around ten steps required to reach the desired compound.

The synthetic route to the above octahydroindole intermediate proposed by Henning et al. (Tett. Lett. 24 (1983), 5343-5346) quickly and elegantly introduces a 1,2-trans configuration around a cyclohexane ring, but requires the use of mercuric nitrate. The use of mercury compounds is obviously undesirable in the preparation of pharmaceuticals. A further synthesis is provided by Brion et al. (Tett. Lett. 33 (1992) 4889-4892) but it is unclear whether they in fact prepare 5% or 95% of the desired product with 2S stereochemistry. In any case, the method requires eleven steps including an initial pig liver esterase digestion to provide the product in stereochemically pure form but in a 95:5 mixture of isomers at position 2. This method is thus complex and ill suited to industrial scale preparation.

In view of the syntheses presently available, there remains a considerable need for methods allowing the synthesis of 2-substituted trans-octahydroindoles suitable for industrial production without the use of hazardous reagents.

The present inventors have now established that 2-substituted trans-octahydroindoles may be synthesised from cyclohexylaziridines in few steps and good yield without the need for hazardous reagents.

In a first aspect, the present invention therefore provides a method for the synthesis of a compound of formula I as a mixture of enantiomers, 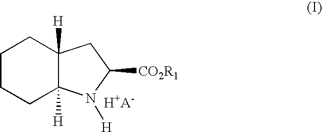
(wherein R₁ is H or an acid protective group and H⁺A⁻ indicates an optional acid with which the compound of formula I may form an ammonium salt)
said method comprising;
A) reacting a cyclohexyl aziridine with a dialkyl malonate, whereby to provide a trans-fused 3-alkylcarbonyl-octahydro-indol-2-one;
B) decarbonylation at the 3-position, conversion of the ketone of the resulting trans-octahydro-indol-2-one to an optionally protected carboxylic acid group; and
C) optionally removing any N-substitution if necessary.

The present inventors have further established that by use of the method for the synthesis of a compound of formula I of the invention, a highly effective method for the synthesis of trandolapril may be provided.

In a second aspect, the present invention therefore provides a method for the formation of a compound of formula III comprising forming a compound of formula I by a method of the invention, followed by;

i) amide formation with an activated acid of formula IV or V;

ii) separation of enantiomers by conversion to diastereoisomers and separation thereof;

iii) removal of any protecting group at R₁ such that R₁ is hydrogen;

wherein steps i) to iii) may be carried out in any order and the conversion to diastereoisomers in step ii) may be by means of the amide formation of step i); 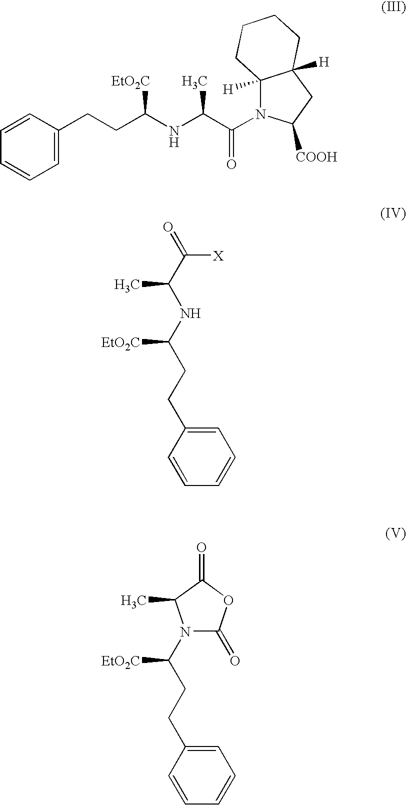
wherein X is OH or an acid activating group.

In the method for the formation of a compound of formula I of the present invention, it is not necessary to provide enantiomerically pure material since the resolution of enantiomers may be more effectively carried out after the compound of formula I has been prepared. It is, however, important that the relative stereochemistry at positions 2, 3a and 7a is provided and in particular that positions 3a and 7a form a trans-ring junction. The present invention provides a method for introducing this trans ring junction with a very high level of control by the ring-opening of a cyclohexyl aziridine compound.

Cyclohexyl aziridine compounds may be prepared, for example, from readily available cyclohexene and N-chlorosulphonamides (chloramines) such as sodium N-chloro p-toluenesulphonamide (“chloramine-T”). This generates an N-substituted cyclohexyl aziridine.

The present inventors have established that ring-opening and re-closing of a cyclohexyl aziridine with a dialkylmalonate, for example of formula VI (wherein R₂ is optionally substituted alkyl, e.g. C₁₋₆ alkyl, such as methyl, ethyl, n-propyl or iso-propyl and may be chiral, such as a menthol derivative), provides 3-alkylcarbonyl-octahydro-indol-2-one in good yield with exclusively the trans ring junction. No cis-fused octahydroindolinone is detected.

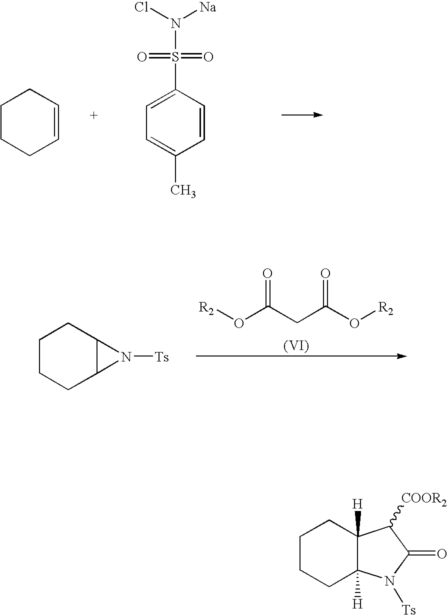

The use of an aziridine in the synthesis of a compound of formula I thus forms a further aspect of the present invention.

Although the generation of one enantiomer of compound I is not necessary in the present invention, it is advantageous if the greater proportion of the product is of the desired (2S, 3aR, 7aS) enantiomer. Typically, the compound of formula I will be the racemate, but the proportion of (2S, 3aR, 7aS):(2R, 3aS, 7aR) compound could be greater than 50:50, preferably at least 60:40, more preferably at least 70:30 and most preferably 80:20 or more. This preferential synthesis of the desired enantiomer may be provided by carrying out the key aziridine ring opening step in the presence of a chiral auxiliary. Examples of suitable chiral auxiliaries are well known in the art and include chiral alkanols such as methanol derivatives, chiral alkyl amines and chiral cyclic amides (such as oxazolidinones). The chiral auxiliaries may be incorporated into the malonate, e.g. as one or more of the R₂ groups thereof or may be present in the reaction medium.

The alkylcarbonyl group generated at position 3 by the reaction of a malonate with the aziridine may be removed, for example by heating in a solution of a halide salt (such as NaCl) in DMF, followed by hydrolysis with water to provide the trans-octahydroindol-2-one.

A highly effective method has been developed by the present inventors for the conversion of a trans-octahydroindol-2-one generated by the method of the invention to the compound of formula I with the desired relative stereochemistry at position 2.

In a preferred aspect, the present invention therefore provides a method for forming a compound of formula I as described herein wherein the conversion of the ketone to an optionally protected carboxylic acid comprises the reduction of said ketone to an alcohol moiety, followed by the stereoselective conversion of said alcohol moiety to a nitrile compound of formula II, followed by conversion of said nitrile compound to an optionally protected carboxylic acid; 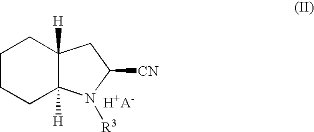
wherein H⁺A⁻ are as defined above and R₃ is H or a leaving group, e.g. tosyl.

Reduction of the trans-octahydroindol-2-one to the trans-octahydroindolin-2-ol may be carried out by established methods, such as the use of diisobutylaluminium hydride (DIBAL). The conversion of the resulting octahydroindolin-2-ol to the corresponding 2-cyano-octahydroindoline may be carried out with trimethylsilylcyanide (TMSCN). Whilst this conversion can be carried out in the presence of a chiral auxiliary (e.g. as hereinbefore described), it has surprisingly been established, however, that when TMSCN is used for this purpose in the presence of a suitable metal salt, such as iron (III), tin or titanium chloride, a very high proportion of product with the desired relative stereochemistry (as shown above for formula II) is generated. The use of TMSCN and a metal salt (especially iron, tin or titanium chloride) to introduce the desired 2S, 3aR or 2R, 3aS stereochemistry in the synthesis of a compound of formula I thus forms a further aspect of the present invention.

Following introduction of the nitrile group (with the correct stereochemistry) at position 2, this may be converted to the carboxylic acid by, for example, treatment with concentrated acid, such as 35% HCl. The conversion method will, in most cases, also remove the N-substituent group remaining from the original aziridine, i.e. the R₃ group (e.g. the p-toluene sulphonate group from N-tosyl aziridine) but this may be removed in a separate step if necessary. If acid treatment is used, the resulting compound of formula I will generally be a salt, where H⁺A⁻ is the acid used for conversion and/or the acid corresponding to the removed N-substituent group (such as tosic acid). Any such H⁺A⁻ may be substituted or removed as desired by use of the appropriate ion exchange resin in well known procedures.

When initially synthesised, the compound of formula I generated by any of the methods of the invention will generally have R₁═H. It will be desirable in some cases, however, to protect the carboxylic acid in formula I prior to further reaction. This allows the reaction of this acid to be better controlled during the later steps towards the synthesis of trandolapril.

Where trandolapril, rather than trandolaprilat, is the desired product, it will be necessary that any protecting group R₁ in formula I is removable without disruption of the carboxyethyl moiety in trandolapril. Thus, suitable protecting groups are those which may be removed without the use of strong aqueous bases and include; groups cleavable with mild acid (such as t-butyl esters), groups cleavable with mild base (such as 9-fluorenylmethyl esters), groups cleavable with fluorinated compounds (particularly silylated compounds including (2-trimethylsilyethoxy)methyl esters which are cleavable with Bu₄NF and TMS ethyl esters which are cleavable with fluoride ion), groups cleavable by photolysis (such as o-nitrobenzyl esters) and groups cleavable by reductive conditions (such as trichloroethyl esters which are cleavable with zinc dust or benzyl esters, which are cleavable by hydrogenolysis at atmospheric pressure). All of the protective ester groups indicated herein, and many others, may be formed by methods well known in the art. This is typically by ester activation and coupling, e.g. by use of a carbodiimide such as dicyclohexylcarbodiimide (DCC), diisobutylcarbodiimide (DIC) or ethyl(dimethylaminopropyl)carbodiimide hydrochloride (EDC). It is preferred to protect the acid moiety as a benzyl ester.

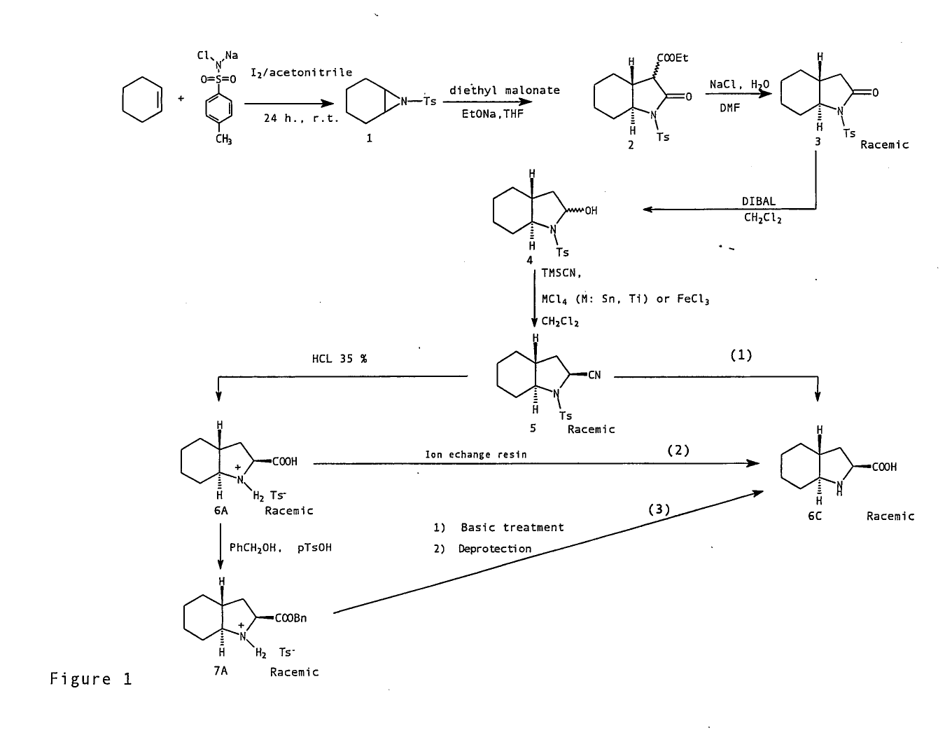

A reaction scheme indicating some of the possible methods for forming compounds of formula I according to methods of the present invention are shown in FIG. 1.

The conversion of the intermediate of formula I to trandolapril may be carried out by the steps of;

i) amide formation with an activated acid of formula IV or V;

ii) separation of enantiomers by conversion to diastereoisomers and separation thereof;

iii) removal of any protecting group at R₁ such that R₁ is hydrogen;

The amide formation step will attach the acid of an activated N-(1-S-ethoxycarbonyl-3-phenylpropyl)-L-alanine derivative of formula IV or V to the nitrogen of the compound of formula I, formed in the method of the present invention.

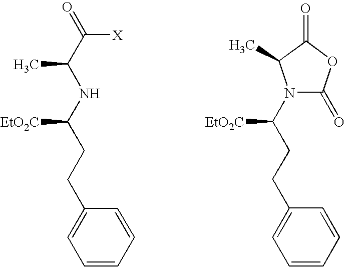

In the compound of formula IV, the acid is activated by the formation of an activated ester. This activation converts the X═OH group to the corresponding compound where X is an acid activating group and may occur in a preactivation step, or may be part of a “one-pot” coupling reaction with the nitrogen of compound I. In such a reaction, the compound of formula IV wherein X is an acid activating group is formed only transiently from, for example the corresponding acid wherein X is OH, in the reaction mixture. An activated ester IV may thus, for example, either be separated and purified, or simply generated in situ. The amide formation reaction will generally be carried out by use of one or more coupling reagents, which will form the activated acid or ester IV, either in a pre-activation step or as part of the coupling step.

Suitable coupling reagents include DCC, DIC and EDC, as well as other common amide forming reagents such as benzotriazol-tris-(dimethylamino)phosphonium hexafluorophbsphate (BOP), O-(7-azabenzotriazole-1-yl)-N,N,N′,N′-tetramethyluronium hexafluorophosphate (HATU), O-benzotriazole-1-yl-N,N,N′,N′-tetramethyluronium hexafluorophosphate (HBTU) and benzotriazole-1-yl-oxy-tris-pyrrolidino-phosphonium hexafluorophosphate (PyBOP).

Where carbodiimide coupling agents are used in “one pot” coupling reactions, the group X in formula IV will be the O-acylisourea product formed by the addition of the acid group to the carbodiimide coupling reagent, which intermediate may also, either fully or partially, react again with another molecule of acid to form the symmetrical anhydride. In the case of the anhydride, the X group of formula IV will be an oxygen bonded to another molecule of formula IV.

Where other coupling reagents are used, X may be a O-pentafluorophenyl group (which is usually stable and can be isolated and then typically reacted in the presence of hydroxybenzotriazole (HOBt)), an O-benzotriazole group or an O-azabenzotriazole group. Obviously, other acid activating groups are well known in the art and may also be used.

Where the amide bond is formed using a compound of formula V, this is a pre-activated anhydride compound and can be used either alone or in combination with other coupling reagents.

The amide formation reaction may be carried out on the compound of formula I as a mixture of enantiomers as formed in the method of the present invention. In such a case, the amide formation with optically pure compound IV or V will result in the formation of separable diastereoisomers. These may then be separated by crystallisation, chromatography or other known methods.

If desired, for example to reduce the amount of compound of formula IV or V consumed in the synthesis, the compound of formula I may be resolved into the desired 2S, 3aR, 7aS enantiomer prior to amide formation. This resolution may be carried out by well established methods including formation or a diastereomeric salt with a stereochemically pure chiral resolving agent such as an acid (or optionally base where base where R₁ is H). A preferred chiral resolving acid is O,O′-dibenzoyl-L-tartaric acid.

Diastereomeric salts may be separated by standard separation methods including crystallisation and chromatography. Amide formation after resolution of the enantiomers of formula I may be carried out as indicated above.

An acid protecting group may be used at position R₁ in the compound of formula I (or the enantiomerically purified compound as considered above). This protecting group will be particularly useful in “one-pot” couplings between compounds of formulae I and IV. In such a reaction, it will be desirable to activate the acid group of the compound of formula IV but not the acid of compound I. Thus, the group R₁ should be a protecting group as considered above.

The acid protecting group R₁ could also be a chiral resolving agent, thereby both protecting the acid moiety in formula I, and allowing resolution of the optical isomers thereof. The other criteria of suitable R₁ groups are discussed above.

Since the protecting group R₁ may be used for protection of the acid and/or for resolution of the isomers, this may be removed at any appropriate stage of the synthesis. Suitable deprotection methods are considered herein above.

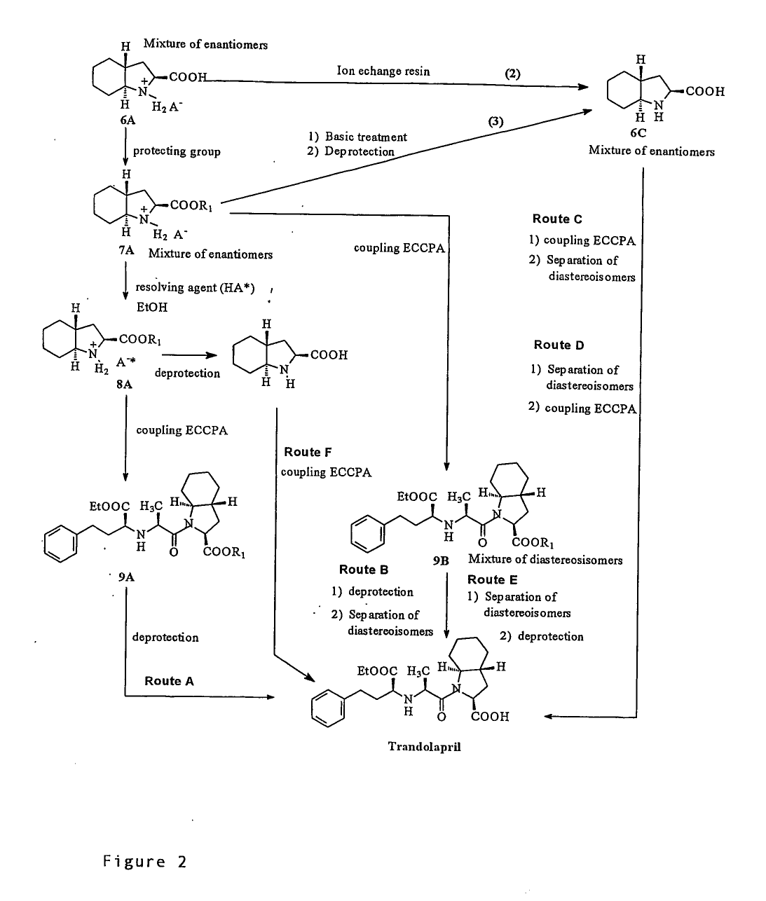

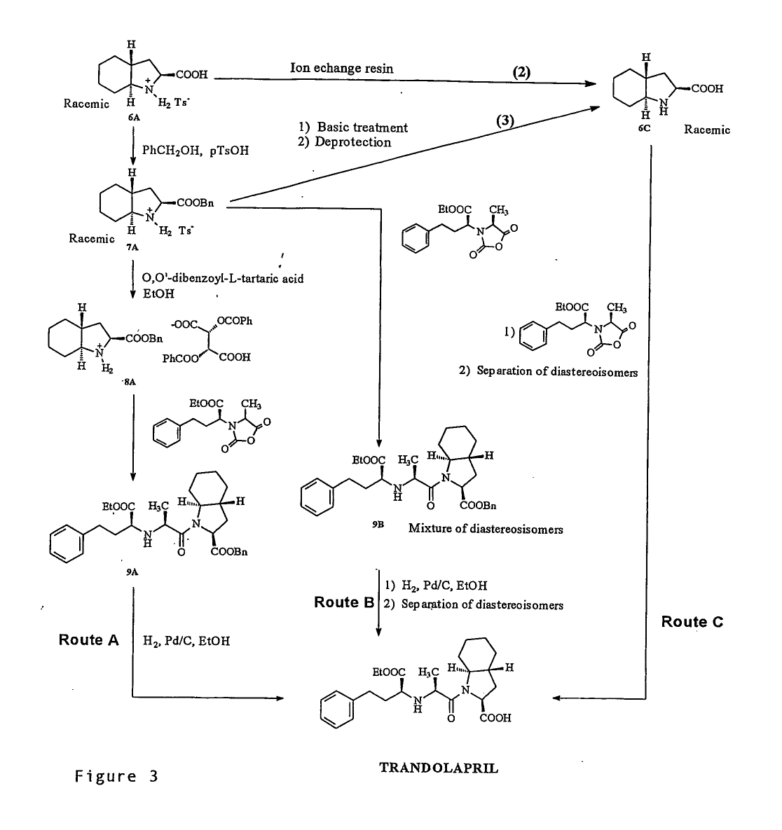

Example synthetic routes to trandolapril from compounds of formula I are summarized in FIG. 2 and include the following routes A-F. In these routes, R₁ is preferably Bn and HA* is preferably O,O′-dibenzoyl-L-tartaric acid. Specific examples of some of these routes are indicated in FIG. 3.

ROUTE A—Separation of enantiomers by the formation of diastereomeric salts with a chiral resolving agent HA* (such as O,O′-dibenzoyl-L-tartaric acid), coupling with N-[1-(S)-ethoxycarbonyl-3-phenylpropyl]-L-alanine (ECPPA) derivative and finally deprotecting the carboxylic acid moiety R₁ (such as by hydrogenating a benzyl ester, where R₁═Bₙ)

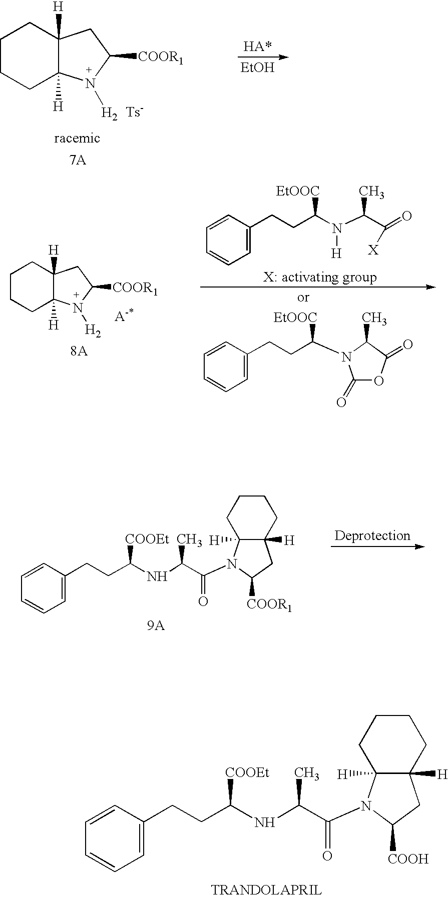

ROUTE B.—Direct reaction of 7A with ECPPA derivative that leads to the formation of diastereoisomers, deprotecting the carboxylic acid moiety and finally separation of diastereoisomers by conventional methods.

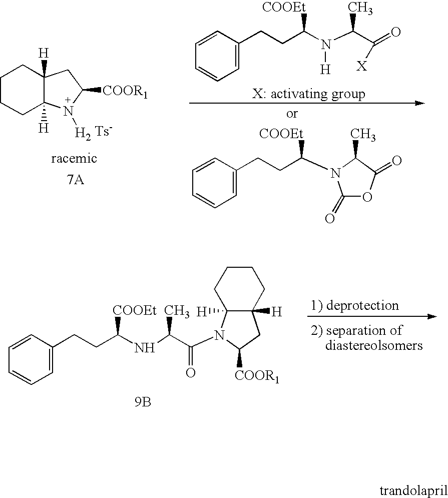

ROUTE C.—Treatment of 7A in basic medium and deprotection that leads to the racemic mixture of octahydroindole acid followed by the reaction with ECPPA derivative. This will result in a diastereomeric mixture that can be separated by conventional methods.

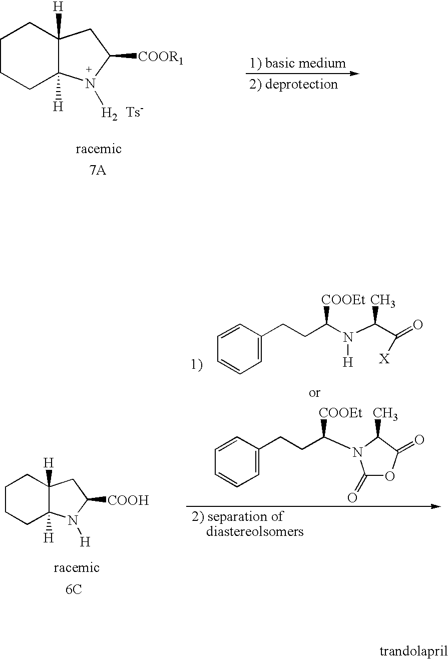

Route D. Separation of isomers of 6C by conventional methods (i.e. formation of a diastereomeric salt) and coupling with ECCPA derivative. 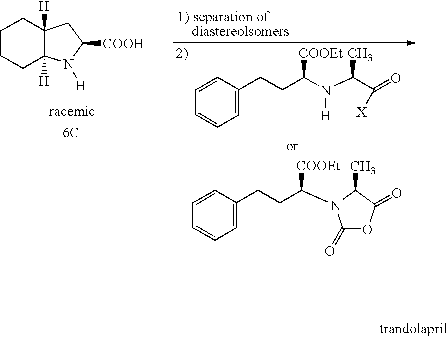
Route E

This route is an inversion of the steps of route B. Firstly the isomers are separated and then the protecting group is removed.

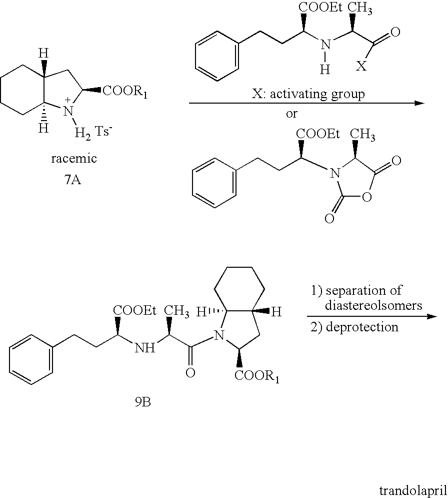

Route F.—The compound 8A is treated to remove the protecting group and coupled with an ECPPA derivative.

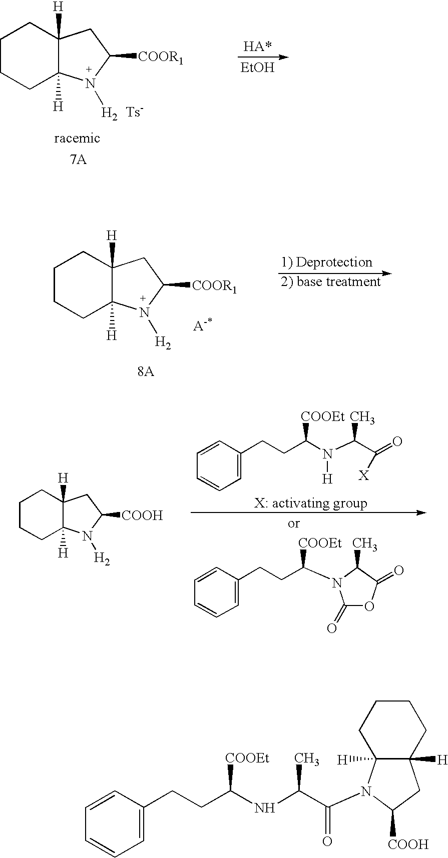

In a yet further aspect, the present invention provides an intermediate or formula I as defined herein formed by the method of the invention.

In a still further aspect, the present invention provides trandolapril formed by the method of the invention.

In a yet still further aspect, the present invention provides the use of trandolapril formed by the method of the invention in the manufacture of a medicament for the treatment of cardiovascular disease. Preferably said cardiovascular disease is hypertension, heart failure or left ventricular disease.

The invention is further illustrated by the following non-limiting Examples, and the attached figures, in which;

FIG. 1 represents a method for the formation of certain compounds of formula I;

FIG. 2 represents some example methods for synthesising trandolapril from various compounds of formula I; and

FIG. 3 represents some preferred methods for synthesising trandolapril from various compounds of formula I.

### EXAMPLES

Abbreviations

DCM Dichloromethane
DIBAL-H Diisobutylaluminium hydride
DMF Dimethylformamide
ECCPA N-[1-(S)-ethoxycarbonyl-3-phenylpropyl]-L-alanine
NCA N-carboxyanhydride
THF Tetrahydrofurane
TMSCN Trimethylsylil cyamide

### Example 1

### Preparation of N-(p-toluenesulfonyl)-[b,c]cyclohexaneaziridine (1) in THF

A mixture of chloramine-T (871.48 g), Iodine (49.67 g) and cyclohexene (800 ml) were dissolved in acetonitrile (2600 ml) and stirred at room temperature for 19 h. The mixture was filtrated through silica-gel and the solvent distilled off. The residue was dissolved in dichloromethane and a solution of sodium thiosulfate and sodium chloride in water. The aqueous phase was extracted with DCM and the combined organic phases were washed with brine and distilled off. The solvent was replaced by tetrahydrofurane, thus obtaining a solution of 1 in THF (88% yield).

### Example 2

### Preparation of N-(p-toluenesulfonyl)-3-ethoxycarbonyloctahydro-1H-indole-2-one (2)

700 ml of diethylmalonate were added to a solution of sodium ethoxyde (315.65 g) in THF (2800 ml) at room temperature under nitrogen atmosphere. The mixture was refluxed for 45 minutes and the solution of 1 prepared in the previous example (3000 ml) was added during 1 hour. After 15 hours at reflux, the mixture was cooled down, diluted with water and the pH adjusted by addition of concentrated hydrochloric acid (110 mL). The organic solvent was distilled off, replaced by toluene (1900 mL) and the aqueous phase separated. The organic phase was washed with brine, and the toluene was eliminated by distillation under reduced pressure. The oil obtained was identified as the title product (73.4% yield).

### Example 3

### Preparation of N-(p-toluenesulfonyl)-octahydro-1H-indole-2-one (3)

A solution of 3.50 g of sodium chloride in 110 mL of DMF, under nitrogen atmosphere, was warmed to 140-145° C. 1.1 mL of water were added and then 17.57 g of 2 in 50 mL of DMF were slowly added during 1.5 h. The solution was stirred while maintaining the temperature between 140 and 145° C. After 18 h the mixture was cooled down to room temperature, 120 mL of water were slowly added during 15 minutes while keeping the temperature below 20° C. Then the solution was cooled below 10° C. for 1 hour and filtered. The solid washed with water and dried under vacuum for 26 h. 11.04 g of 3 were obtained as a yellowish solid (66.5% yield).

### Example 4

### Preparation of N-(p-toluenesulfonyl)-2-hydroxyoctahydro-1H-indole (4)

A solution of DIBAL-H (1M in hexanes, 1262.4 g) was slowly added (3 hours) to a solution of 3 (420 g) in dichloromethane (2100 mL) cooled at −5° C. The mixture was stirred for 50 minutes and methanol and water were slowly added. The two phases were separated and the organic phase was filtered through celite and evaporated to dryness to yield a white solid (93% yield).

### Example 5

### Preparation of (2S*, 3aR*, 7aS*)-N-(p-toluenesulfonyl)-2-cyanooctahydro-1H-indole (5)

To a solution of 4 (390 g) in dichloromethane (1500 ml) cooled at −5° C., were added TMSCN (290 ml) and 208.28 g of SnCl₄ (1M in DCM). The mixture was stirred for 3 hours while maintaining the temperature at −5° C. The mixture washed with a solution of sodium carbonate, the aqueous phase was extracted twice with dichloromethane. The collected organic phases were filtered through Celite and the solvent was distilled off. The residue was suspended in methanol for 1 hour at 40° C., and 1 hour at 5-10° C. 321.63 g of 5 were obtained as a white solid by filtration (78.1% yield, SSR isomer: 4.8%).

### Example 6

### Preparation of (2S*, 3aR*, 7aS*)-octahydro-1H-indole-2-carboxylic acid (6A)

To a suspension of 150.07 g of 5 in 1000 mL of HCl conc. were added 1000 mL of HCl conc. and 750 ml acetic acid glacial. The mixture was refluxed (Ti=100° C.) for 1 hour approx., until the initial violence of the reaction was controlled. Then 1750 ml of HCl conc, were added in 30 min (without stopping reflux) and reflux was gone on for 19 more hours. Then it was cooled down to room temperature. The resulting aqueous phase washed twice with 750 mL of toluene and filtered. The product is obtained as an aqueous solution of hydrochloride and tosylate of 6A. (Quantification by HPLC: 81.04 g of 6, 97.0% yield, SSR isomer 6.1%).

### Example 7

### Preparation of benzyl (2S*, 3aR*, 7aS*)-octahydro-1H-indole-2-carboxylate (7A)

2980 mL (53 g) of the aqueous solution of 6A, prepared in the previous example, were distilled off under reduced pressure to a total volume of 100 mL. Then, portions of 100 mL of toluene were successively added and distilled off until the Karl-Fischer of the solution was approximately 0.09%. 900 mL of toluene, 95.4 g of p-toluenesulfonic acid and 119.2 mL of benzyl alcohol were added to the suspension. The water was eliminated by azeotropic distillation during 15 minutes. The amount of toluene distilled was replaced by fresh toluene, and the suspension was cooled down to 45° C. and filtered through a sintered funnel, the solid washed with 340 mL of toluene. The filtrated was warmed to 110° C. and the water was eliminated by azeotropic distillation during 2 h. Then the necessary toluene was added until 500 mL. The solution was cooled to 20-25° C. and seeded with 7A. 131 g of a solid which contains a mixture of hydrochloride and tosylate of 7A was obtained (61.6 g of 7A quantified by HPLC, 76% yield, 2.5% SSR). This solid was recrystallized in 600 mL of toluene and 21 mL of ethanol that yielded 119.5 g of solid containing 57 g of free 7A (by HPLC) (92.6% yield, 0.6% SSR).

### Example 8

### Preparation of benzyl (2S, 3aR, 7aS)-octahydro-1H-indole-2-carboxylate O,O′-dibenzoyl-L-tartrate (8A).

To a suspension of 7A obtained in the previous example (64 g, 30.5 g of free 7A) in DCM (150 mL) were added 69 mL of water and a saturated solution of sodium carbonate (120 mL). The organic phase was separated and the aqueous phase extracted with 2×80 mL of DCM. The organic phases were collected, filtered and evaporated to dryness to yield 7A as a free base.

This residue was dissolved in 90 mL of absolute ethanol, and a solution of O,O′-dibenzoyl-L-tartaric acid (29.8 g) in 525 mL of absolute ethanol was added. The mixture was stirred for 3 hours at room temperature. The solid was filtered and washed with 2×100 mL of absolute ethanol. 26.36 g (36.30 yield) of 8A were obtained (SSR: 0.5%, RSR: 0.8%).

### Example 9

### Preparation of (2S, 3aR, 7aS)-1-[(S)—N—[(S)-Carboxy-3-phenylpropyl]alanyl] octahydro-1H-indole-2-carboxylic acid, 1-ethyl ester (Trandolapril)

From 8A

To a suspension of 21.6 g of 8A in DCM were added 108 mL of aqueous NaOH (1N) and the solution was stirred for 15 minutes. The two phases were separated and the aqueous layer washed twice with 100 mL of DCM. The collected organic phases were filtered and the solvent evaporated to dryness.

11.1 g of ECCPA-N-carboxyanhydride (ECCPA-NCA), in 35 ml DCM were added to the solution of 8A in 30 mL of DCM under inert atmosphere and stirred at room temperature for 4 hours. 62 mL of a saturated solution of sodium bicarbonate were added to the mixture and stirred for 15 minutes. The separated aqueous layer washed with 2×50 mL of DCM. The collected organic phases were washed with 62 mL of distilled water, filtered and evaporated to dryness.

A solution of the residue obtained in 90 mL of ethanol was vigorously stirred at 10° C. under hydrogen atmosphere (P=1.5 bar) in presence of 1.54 g of Pd/C 10% (humidity 59%). After 90 minutes the catalyst was filtered off through Celite and washed with ethanol. The solvent was evaporated to dryness.

To the residue obtained were added 150 mL of diisopropylether and 6 mL of ethanol, and a solid precipitated. The suspension was stirred for 1 hour at room temperature and the solid was filtered off and was washed with 2×20 mL of diisopropylether. 12.35 g of Trandolapril were obtained (82% yield, HPLC: 0.2% of SSSSR isomer).

From 7A

To a suspension of 44.7 g of 7A in 105 mL of DCM were added 49.5 mL of distilled water and a saturated solution of sodium carbonate (82.5 mL) and the solution was stirred for 15 minutes. The two phases were separated and the aqueous layer washed twice with 60 mL of DCM. The collected organic phases were filtered and the solvent evaporated to dryness.

26.9 g of ECCPA-N-carboxyanhydride (ECCPA-NCA), in 65 mL of DCM were added to the solution of 7A in 46 mL of DCM under inert atmosphere and stirred at room temperature for 4 hours. 100 mL of a saturated solution of sodium bicarbonate were added to the mixture and stirred for 15 minutes. The separated aqueous layer washed with 2×50 mL of DCM. The collected organic phases were washed with 100 mL of distilled water, filtered and evaporated to dryness.

A solution of the crude obtained in 176 mL of ethanol was vigorously stirred at 10° C. under hydrogen atmosphere (P=1.5 bar) in presence of 3.76 g of Pd/C 10% (humidity 59%). After 5 hours the catalyst was filtered off through a Celite path and washed with ethanol. The solvent was evaporated to dryness.

To the residue obtained were added 365 mL of diisopropylether and 18.2 mL of ethanol, and a solid precipitated. The suspension was stirred for 1 hour at room temperature and the solid was filtered off and was washed with 2×30 mL of diisopropylether. 15.4 g of Trandolapril were obtained (42% yield, HPLC: 0.06% of SSSSR, 0.28% SSRSR, 0.16% unknown isomer, 0.19% DKP).

This solid was purified by suspending it in a mixture of 77 mL of diisopropylether and 15.4 mL of ethanol, and stirring the suspension at 40° C. for 1 hour and at 0-5° C. for 1 hour more. The solid was filtered off and washed twice with 20 mL of diisopropylether. After this purification 15 g of Trandolapril were obtained (97% yield) (HPLC: SSSSR 0.04%, SSRSR 0.09%, 0.09% unknown isomer).

1. A method for the synthesis of a compound of formula I as a mixture of enantiomers, 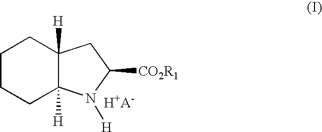
(wherein R₁ is H or an acid protective group and H⁺A⁻ indicates an optional acid with which the compound of formula I may form an ammonium salt)
said method comprising;
A) reacting a cyclohexyl aziridine with a dialkyl malonate, whereby to provide a trans-fused 3-alkylcarbonyl-octahydro-indol-2-one;
B) decarbonylation at the 3-position, conversion of the ketone of the resulting trans-octahydro-indol-2-one to an optionally protected carboxylic acid group; and
C) optionally removing any N-substitution if necessary.

2. A method as claimed in claim 1 wherein said mixture of enantiomers consists of more than 50:50 (2S, 3aR, 7aS):(2R, 3aS, 7aR).

3. A method as claimed in claim 2 wherein step A and/or step B is carried out in the presence of a chiral auxiliary.

4. A method as claimed in claim 1 wherein said cyclohexyl aziridine is

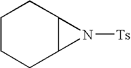

5. A method as claimed in claim 1 wherein said dialkylmalonate is diethylmalonate.

6. A method as claimed in claim 1 wherein decarbonylation is carried out by heating said trans-fused 3-alkylcarbonyl-octahydro-indol-2-one in the presence of a halide salt and subsequently hydrolysing.

7. A method as claimed in claim 1 wherein conversion of said ketone to an optionally protected carboxylic acid comprises the reduction of said ketone to an alcohol moiety, followed by the stereoselective conversion of said alcohol moiety to a nitrile compound of formula II, followed by conversion of said nitrile compound to an optionally protected carboxylic acid; 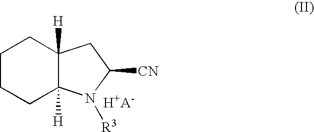
wherein H⁺A⁻ are as defined in claim 1 and R₃ is H or a leaving group.

8. A method as claimed in claim 7 wherein said stereoselective conversion of said alcohol moiety to a nitrile compound of formula II is carried out in the presence of a metal halide.

9. A method as claimed in claim 8 wherein said metal halide is tin tetrachloride, titanium tetrachloride or iron trichloride.

10. A method for the formation of a compound of formula III comprising forming a compound of formula I by a method as claimed in claim 1 followed by;
i) amide formation with an activated acid of formula IV or V;
ii) separation of enantiomers by conversion to diastereoisomers and separation thereof;
iii) removal of any protecting group at R₁ such that R₁ is hydrogen;
wherein steps i) to iii) may be carried out in any order and the conversion to diastereoisomers in step ii) may be by means of the amide formation of step i); 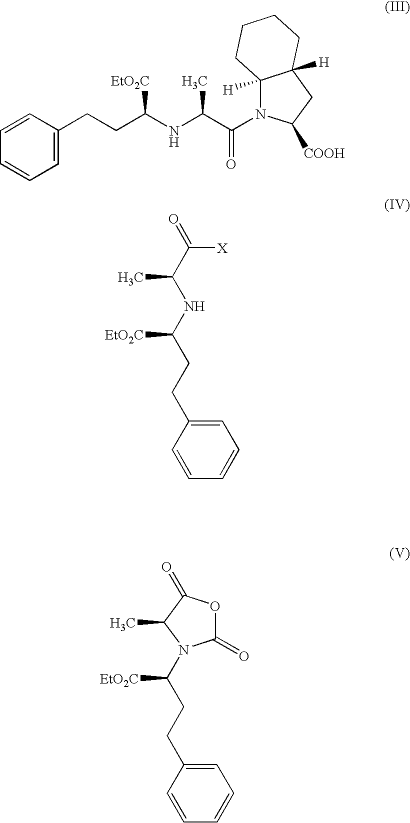
wherein X is OH or an acid activating group.

11. A method as claimed in claim 10 wherein the protecting group at R₁, if present, is a benzyl group.

12. A method as claimed in claim 10 wherein diastereomer separation is carried out via reaction with O,O′-dibenzoyl-L-tartaric acid.

13. A method as claimed in claim 10 wherein the steps are carried out in the order (i), (iii) and (ii).

14. A method as claimed in claim 10 wherein the steps are carried out in the order (ii), (i) and (iii).

15. An intermediate of formula I as defined in claim 1, formed by a method as defined above.

16. An intermediate of formula II as defined in claim 7, formed by a method as defined above.

17. Trandolapril having formula III as defined in claim 10 formed by a method as defined above.
